(19) United States
(12) Patent Application Publication
(10) Pub. No.: US 2011/0091947 A1
(43) Pub. Date: Apr. 21, 2011

(54) High-Purity Purification Method for Omega-3 Highly Unsaturated Fatty Acids

(75) Inventors: Gap Jin Kim, Daejeon (KR); Hong Joo Son, Busan (KR); Wu Song Whang, Daejeon (KR); Yoon Mo Koo, Incheon (KR); Jin IL Kim, Incheon (KR); Jin Hyo Yang, Incheon (KR)

(73) Assignee: AK BIOTECH CO., LTD., Daejeon (KR)

(21) Appl. No.: 12/999,177

(22) PCT Filed: Jun. 3, 2009
(86) PCT No.: PCT/KR09/02958

§ 371 Date: Dec. 15, 2010

(30) Foreign Application Priority Data
Jun. 20, 2008 (KR) 10-2008-0058114

(51) Int. Cl. C12P 7/64

(52) U.S. Cl. 435/134

(57) ABSTRACT

The present invention relates a high-purity purification method for omega-3 highly unsaturated fatty acids. More specifically, it relates to a high-purity purification method for omega-3 highly unsaturated fatty acids which is both environmentally friendly and easy to implement and comprises the steps of: a) preparing a fatty acid ethyl ester (FAEE) by ethanolysis of a natural oil or fat, using ethanol, in the presence of an enzyme catalyst extracted from at least one microorganism selected from the group consisting of Candida genus, Rhizopus genus, Mucor genus, Aspergillus genus and Pseudomonas genus; b) subjecting the said prepared fatty acid ethyl ester to preliminary distillation using short-path distillation (SPD) device between 100 and 200° C. and between 0.001 and 10 mmHg; c) forming a concentrated fatty acid by subjecting the ethyl ester, which has been subjected to the preliminary distillation, to reduced-pressure fractional distillation at between 100 and 200° C. and between 0.001 and 10 mmHg; and d) purifying the concentrated fatty acid by means of simulated moving bed (SMB) column chromatography.

### TECHNICAL FIELD

The present invention relates to a high-purity purification method for omega-3 highly unsaturated fatty acids, and more specifically, to an environmentally friendly high-purity concentration or purification method for omega-3 fatty acid based highly unsaturated fatty acids which are one of the essential fatty acids originating from fish oil.

### BACKGROUND ART

Highly unsaturated fatty acids are the fatty acids that have two or more double bonds in their molecular structure. Their importance has recently been realized and is now named as “essential” fatty acids with “essential” amino acids, “essential” vitamins and the like, because it has proven to be effective in decreasing cholesterol and preventing and treating diseases of the circulatory system such as arteriolosclerosis.

These include mainly linolic acid or linoleic acid in the seed oil such as safflower oil, soybean oil, sunflower seed oil, corn oil, perilla oil, and the like, or nuts, alpha linolenic acid in flaxseed oil or perilla, gamma linolenic acid in evening primrose oil, EPA, DHA, and the like in mainly the oily fish such as Pacific saury, sardine, mackerel, tuna, and the like, and arachidonic acid directly obtained from the embryoid of a recent microorganism or microalgae.

These fatty acids are representative of highly unsaturated fatty acids having double bonds, ranging from two to six, and are widely used ranging in terms of the recent health functional nutraceutical food material to the medicinal material. Further, their recognition has rapidly expanded from Northern Europe such as Sweden, Denmark, and the like, to the Middle East and China via North America such as Canada and USA, and is widely recognized as one of the foods that improve the QOL (Quality of Life).

Among them, Omacor™, a high-purity DHA-containing product made by a Norwegian based Pronova Company, is a secondary prevention drug after post-hypertriglyceridemia and post-myocardial infarction, and the most recently approved ethical drug from the FDA, because of the confirmed results of high effectiveness of reducing the numerical value of triglyceride by 45%, and reducing the cardiovascular disease death rate by 30% or more when taking it for the purpose of secondary prevention after post-myocardial infarction.

Specifically, because Omacor™ is an ethical drug being purified and concentrated from fish oil, in clinical trials, its big advantage has found that the stability and drug tolerance are excellent and there is no drug interaction when combining medication with other drugs administrated for the purpose of treating hypercholesteremia, diabetes and the like, which is common in a hypertriglyceridemia patient.

Omacor™ is the first Omega-3 fatty acids products which was approved by the FDA as an ethical drug in November 2004, launched October 2005 in the US under the same product name and got an explosive response in its first 10 weeks, as a potential blockbuster. Further, it has been approved in most of Europe and Asia, is in alliance with Pfizer, SPA, Sigma TAU, Solvay, Astrazeneca, and is now on the market by Takeda Pharmaceutical in Japan, and Kuhnil Pharmaceutical in Korea.

Even though the highly unsaturated fatty acids such as DHA are contained in mainly the fish oil, 20˜25% of the fish oil are contained in the tuna's eyeball oil. In Northern Europe such as Norway, and the like use salmon oil, Japan uses tuna oil, while Korea imports most of the raw materials from Canada, Australia, Japan, South-East Asia (Thailand, Malaysia, Philippines), and the like.

However, these fatty acids are easily oxidized in the air, which generate peroxides and are polymerized. Consequently, it smells bad in the fish oil, which is caused by a material generated due to the oxidization and degradation of highly unsaturated acid contained in the fish oil. Therefore, a separate pre-treatment method and special purification process in order to separate these fatty acids from the fish oil, concentrate and purify them, are needed.

Generally, raw oil being taken from animals and plants contain various impurities. The impurities include, for example saccharide, protein and its hydrolysate, phospholipid, sterin, tocopherol, pigment, slime, fatty acids, and the like, preferably tocopherol which is natural antioxidant. Meanwhile, phospholipid, saccharide, protein, slime, and the like color the oil such as dark fat color, or fume or bubble during the course of processing the oil or fat, should be removed in advance. In addition, since free fatty acids make acid value high and decreases the quality of the oil or fat and instead makes soap, these should be removed in advance.

In addition to the operation to remove undesirable impurities in advance, which is generally referred to as refining, these important operations are also needed, including degumming, refining, bleaching, deodorization, and the like.

The oil or fat in nature is present mainly in the form of triglyceride (TG), which is when three molecules of fatty acids are coupled with one molecule of glycerin. In order for high-purity purification of them, a process for preparing alkyl ester of fatty acids and removing glycerin via the ester exchange reaction with alcohol oil is needed.

The example of the ester exchange method using alcohols include a method of using alkali as a catalyst, a method of using lipase lipolysis enzyme or supercritical fluid (methanol, ethanol), and the like. Currently an alkali catalyst method is the most common method. However, in the method for preparing fatty acid alkyl ester by using the existing alkali catalyst, since the catalyst is coupled with free fatty acids to generate fatty acid soap as a by-product, an excessive amount of alkali catalyst is needed, and the yield is decreased. In addition, it is difficult to separate fatty acid alkyl ester layer that is formed as a by-product from a glycerin layer, and an additional process for removing the catalyst and fatty acid soap is needed.

For resolving these problems, the environmentally friendly process such as the alcoholysis method using microorganism-originated lipase and alcohol as an enzyme catalyst not using an alkali catalyst, and the method for preparing alkylester by applying a supercritical process using alcohol in the supercritical or subcritical condition, and the like are actively researched and developed around the biodiesel-producing industry.

Previously, there was an example of preparing 95% high-purity DHA from tuna oil by using supercritical fluid chromatography (SFC) as the environmentally friendly process (Alkio, and the like, 1999), however the cost was $550/kg which is too expensive to be commercialized, and the method of using lipase enzyme catalyst was not widely used due to the high enzyme price and the inactivation problem of enzyme under reaction. However, a biodiesel producing technique is actively being developed due to the increase in environmental problems; and as a result, the process for preparing alkylester by using an enzyme catalyst is considered as an environmentally friendly alternative process.

Korean Patent Registration No. 139006 is very similar to Japanese laid-open patent publication (No. Sho 58-8037), and uses as alcoholysis catalyst, however, sodium ethoxide is a flammable and toxic material; consequently, its use for food preparation is forbidden in advanced country. In addition, by using this catalyst, the process causes environmental pollution by generating toxic waste water due to washing the reactant. Furthermore, it was reported that the purity of unsaturated fatty acid obtained through continuous distillation at 195° C.˜208° C. is only 30˜60%, and the reason why the distillation temperature is high is form nonvolatile decomposition products, that is, a structural isomer of polymer, CFAM (cyclic fatty acid monomers), EPA and DHA, (European Journal of Lipid Science and Technology ISSN 1438-7697, 2006).

Japanese laid-open patent publication No. 1999-246888 discloses a method for producing products at 85% or more by using a continuous distillation using a distillation column having 3 steps and an urea adduct method that contacts the main component with urea methanol solution. In addition, Japanese laid-open patent publication No. 1997-302380 proposes the method for producing 85% products by the urea adduct method or producing 98.5% or more products via an Ag acetate treatment after preparing the EPA at a concentration of 80% or more using the method of removing carbon number below C19 and recovering carbon number C21 or more by vacuum distillation that uses a two step distillation or a three step distillation column.

However, such procedures have problems that include a toxic catalyst or the generation of an amount of waste water at the pretreatment process, and the residue of solvent. In addition, improvements in the distillation yield and the productivity are followed by the direct vacuum fractional distillation treatment without the preliminary distillation step of fatty acid esters.

Meanwhile, in Japanese patent No. 3614177, the method comprising vacuum or reduced-pressure distilling fatty acids or ester mixture obtained from natural oil or fat including DHA or their derivative under high vacuum according to the multiple distillation columns, obtaining the middle distillate of which the main ingredient is fatty acid of carbon number 22 or the ester, and preparing high-purity DHA by fraction and purification of them through partition column chromatography has been proposed. In the above, the method for obtaining fatty acids or ester mixture for removing impurities from natural oil or fat is not disclosed, and there is a problem that pollutants like heavy metal and PCBs contained in natural oil or fat are not removed.

Hence, in Japanese laid-open patent publication No. 1996-100191, the method for recovering high-purity DHA ester by esterificating fatty acid mixture obtained from natural oil or fat containing EPA and DHA to lower alcohol to react with urea, separating urea crystal from liquid phase by means of filtering out and centrifuging, etc., fractional distilling and recovering the extracted solvent by heating the separated solvent under vacuum, and reduced-pressure distilling them has been proposed. However, in the method for recovering the high-purity DHA ester, saturated fatty acids or impurities not reacted with urea may still remain in the materials fracted and recovered by performing urea reaction in the beginning.

In addition, in these prior arts, the process for alcoholysis and esterification of fatty acids is applied only to the preparation of fatty acid alkylester for simply preparing biodiesel, and the preparation of fatty acids through the distillation process is mostly the process for the production of eicosapentaenoic acid (EPA). There is no prior art to provide a method for producing ethylester of fatty acids by the enzyme catalyst method being proposed in the present invention, and producing high-purity EPA or DHA of 99% or more through the continuous process of simulated moving bed chromatography via the preliminary distillation using short-path distillation (SPD) device and the reduced-pressure fractional distillation at a distillation temperature of 180° C. or less that minimizes the production of nonvolatile pyrolysis product.

### DISCLOSURE

### Technical Problem

An object of the present invention is to provide a high-purity purification method for omega-3 highly unsaturated fatty acids which environmentally friendly minimizes the generation of waste water, without using a toxic catalyst or caustic soda.

Another object of the present invention is to provide a high-purity purification method including: performing a preliminary distillation using a short-path distillation device to obtain a distilled raw material having improved productivity and without heavy metal and PCBs contained in a natural oil or fat; and minimizing formation of trans isomer of omega-3 fatty acids, a polymer, and cyclic fatty monomers (CFAM) which are pyrolysis products of long-chain highly unsaturated fatty acids by performing a low temperature reduced-pressure fractional distillation at 180° C. or less using the distilled raw material, thereby obtaining producing 99% or more of purity products of the Active pharmaceutical ingredient (API) level during a final chromatography process.

### Technical Solution

In one general aspect, a high-purity purification method for omega-3 highly unsaturated fatty acids includes: a) preparing a fatty acid ethyl ester (FAEE) by ethanolysis of a natural oil or fat, using ethanol, in the presence of an enzyme catalyst extracted from at least one microorganism selected from the group consisting of Candida genus, Rhizopus genus, Mucor genus, Aspergillus genus and Pseudomonas genus; b) subjecting the said prepared fatty acid ethyl ester to preliminary distillation using a short-path distillation (SPD) device at between 100 and 180° C. and between 0.005 and 10 mmHg; c) forming a concentrated fatty acid by subjecting the ethyl ester, which has been subjected to the preliminary distillation, to reduced-pressure fractional distillation at between 150 and 180° C. and between 0.001 and 10 mmHg; and d) purifying the concentrated fatty acid by means of simulated moving bed (SMB) chromatography.

In addition, the present invention is to provide a high-purity purification method for omega-3 highly unsaturated fatty acid which includes lipase having 1,3-positional specificity to triglycerol carbon of natural oil or fat and lipase having acyl chain specificity to triacylglycerol of natural oil or fat as an enzyme catalyst.

In addition, the present invention is to provide a high-purity purification method for omega-3 highly unsaturated fatty acid which 1,3-positional specificity lipase is at least one lipase selected from the group consisting of Rhizopus javanicus, Rhizopus niveus or Aspergillus niger and acyl chain specificity lipase is at least one lipase selected from the group consisting of Candida cylindracea, Candida antarctica, Rhizopus miehei or Rizopus arrhizus.

The purified omega-3 highly unsaturated fatty acid is EPA (Eicosapentaenoic Acid) or DHA (Docohexaenoic Acid), and the present invention provides a high-purity purification method for omega-3 highly unsaturated fatty acid, which has a concentration of 90% or more.

Hereinafter, preferable exemplary embodiments of the present invention will now be described in detail with reference to the accompanying drawings. First, it should be noted that the same components or parts represent the same reference symbol as much as possible. For the description of the present invention, the concrete description about the concern notified function or constitution is emitted in order not to obscure the substance of the present invention.

The terms of “about”, “substantially”, etc., in the present specification are used as a meaning close to or in the numbers when unique preparation and material tolerance are provided to the mentioned meaning, and are used to prevent an unscrupulous infringer from unfairly using the contents of the description where exact or absolute numbers are referred to help understanding this invention.

FIG. 1 is a purification process view of highly unsaturated fatty acid according to an embodiment of the present invention. Referring to FIG. 1, the purification of the highly unsaturated fatty acid of the present invention may be proceeded with the steps of preparation of fatty acid alkyl ester S100, preliminary distillation S200, reduced-pressure fractional distillation S300 and chromatography purification S400.

First, in the preparation of fatty acid alkyl ester S100, step a) preparing a fatty acid ethyl ester (FAEE) by ethanolysis of a natural oil or fat, using ethanol, in the presence of an enzyme catalyst extracted from at least one microorganism selected from the group consisting of Candida genus, Rhizopus genus, Mucor genus, Aspergillus genus, Pseudomonas genus or the mixture thereof may be carried out.

The natural oil or fat being used in the present invention includes fish oil or fat, preferably imported tuna oil or fat or sardine oil or fat, but are not limited thereto.

The natural oil or fat is the material being pretreated by removing the impurities such as phospholipid, saccharide, protein, mucilages, and the like in advance which becomes the cause of coloring, fuming or bubbling of the natural oil or fat when analyzing it with the method selected from the group consisting of degumming, refining, bleaching, deodorization and the combined method thereof.

The natural oil or fat and ethanol may be preferably reacted, while having molar ratio of 3:1˜45:10, the reaction time of 2 to 48 hours, and the ester conversion yield of 80 to 97%.

More specifically, at least one lipase enzyme catalyst having 1,3-positional specificity and extracted from at least one microorganism selected from the group consisting of Candida genus, Rhizopus genus, Mucor genus, Aspergillus genus or Pseudomonas genus, and at least one lipase enzyme having the acyl chain specificity are agitated and mixed with the natural oil or fat. The ethanol is slowly added while stirring the above mixture, and preferably added in a large of amount in accordance with the progress of the reaction so that the molar ratio of natural oil or fat and ethanol is 3:1 to 45:10.

The enzyme catalyst is added in the amount of 0.1 to 10 parts by weight based on 100 parts by weight of the natural oil or fat and ethanol, and then the ester exchange reaction agitating at 100 to 200 rpm at 40±2° C. is practiced to prepare fatty acid ethyl esters.

The enzyme catalyst being used in the present invention is an enzyme being extracted from at least one microorganism selected from the group consisting of Candida genus, Rhizopus genus, Mucor genus, Aspergillus genus or Pseudomonas genus. 1,3-positional specificity lipase of triglycerol carbon in natural oil or fat among the enzyme catalyst is an enzyme which hydrolyzes by reacting only at the 1st and 3rd positions of triglycerol carbon in natural oil or fat. Acyl chain specific lipase is an enzyme catalyst representing the specificity to the carbon number length of fatty acids. The acyl chain specific lipase is also referred to as triacylglycerol hydrolase, and this enzyme is preferably fixed to the specific carrier for continuous maintenance of activity.

The 1,3-positional specific enzyme catalyst may be selected from the group consisting of Rhizopus javanicus, Rhizopus niveus and Aspergillus niger, acyl chain specific lipase may be selected from the group consisting of Candida cylindracea, Candida Antarctica, Rhizopus miehei and Rhizopus arrhizus.

The present invention is to prepare fatty acid ethyl esters by alcoholysis by reacting with a spirit alcohol using the enzyme catalyst.

If the chemical catalyst is used, the reaction occurs at a high temperature, such that cis-trans isomerization and the transition of double bond may occur in a carbon chain of fatty acid. However, if the enzyme catalyst is used, the reaction does not occur at a high temperature, such that side reactions such as cis-trans isomerization and the transition of double bond, etc., do not occur in a carbon chain of fatty acid and as a result, omega-3 fatty acids having a cis structure in the fatty acid may be formed.

The omega-3 fatty acid is naturally formed in a cis structure. However, the fatty acid having a trans structure is generated in the course of processing a cis-type fatty acids having high unsaturation as omega-3. The fatty acid is too stable fatty acid in which a carbon chain of fat is symmetrically (trans-type) formed across the double bonds between carbons of unsaturated fatty acids. It is too stable that even the metabolism is low and which further became a cause of arterosclerosis, heart disease and cardiovascular disease in the cases of being intaken.

Therefore, the present invention is to provide reaction for preventing the transition into this trans structure.

Meanwhile, the ethanol being used in alkylester is preferably a spirit ethanol having the purity of 95% or more. In the case of methyl ester using methanol, ethyl ester is preferred because the metabolism toxicity of methane which is a decomposition product, is becoming a problem.

Thereafter, as a preliminary distillation step S200, step b) subjecting the said prepared fatty acid alkyl ester to preliminary distillation using the short-path distillation (SPD) device between 100 and 200° C. and between 0.001 and 10 mmHg may be carried out. In the preliminary distillation having a condition in which a temperature is below 100° C. and pressure is below 0.001 mmHg, the compound exceeding 100° C. does not evaporate such that the distillation yield may be lowered. When the preliminary distillation condition exceeds 200° C. and 10 mmHg, the temperature and pressure are too high, such that heat denaturated products may be formed.

In other words, the fatty acid ethyl ester being prepared in step a) is continuously concentrated and distilled by using a short-path distillation (SPD) device, and this reaction is practiced at 100˜180° C., and vacuum of 0.001 to 1.0 mmHg. The reaction may be preferably carried out so that the final recovering yield of the distillate is 50 to 70%.

The preliminary distillation step is proceeded before the vacuum distillation. The step is to improve yield and prevent the heat denatured product from being generated at the time of reduced-pressure fractional distillation by previously removing a low molecular material of low boiling point prior to the fractional distillation performed between 100 go 180° C. If the vacuum distillation is practiced without preliminary distillation, a great quantity of low-boiling point compounds are evaporated at once, the degree of vacuum in the reduced-pressure distillation device is hardly maintained, such that it is difficult to carry out continuous distillation under equilibrium condition. In addition, the heat denatured products are highly likely to be found at a high temperature, thereby causing a result that the distillation yield and the quality of the products are degraded.

The short path distillation (SPD) device being used in the preliminary distillation of the present invention includes MYERS-VACUUM, INCON, CHEMTECH SERVICE, ASAHI, ULVAC OR VTA, UIC PRODUCTS, however is not always limited thereto.

In addition, the short path distillation (SPD) device of the present invention is the device which has short distance between evaporation area and condensation area, and is possible to evaporate and concentrate large amounts of heat-unstable material in a short time and separate objects from the other material molecule without collision, by the way of distillation performed under vacuum (0.001˜10 mmHg) at a relatively low temperature (100˜200° C.).

In addition, since all processes, particularly evaporation is carried out under relatively high vacuum, very thin film (thickness 0.1 mm) is formed and contact with heat is minimized. The contact time with heat is only 1˜3 seconds, which is very short, and a low-boiling point compound may be removed before high-vacuum reduced-pressure distillation, and the heat-unstable material may be produced on a large scale.

Therefore, in the present invention, the short-path distillation (SPD) is used to perform rapid evaporation by providing the maximum surface area per unit volume, control the contact time of the solution to several seconds or less for the increased temperature of the surface of the wall, thereby minimizing the destruction or damage of the material such that is sensitive to heat denaturation and oxidation as fatty acid.

The fractional distillation step S300, that is, step c) forming a concentrated fatty acid by subjecting the alkyl ester, which has been subjected to the preliminary distillation, to reduced-pressure fractional distillation at between 100 and 200° C. and between 0.001 and 10 mmHg, may be carried out.

If the fractional distillation is carried out at 100° C. or below 0.001 mmHg, the effect of the fractional distillation is low, however if it is carried out at 200° C. or over 10 mmHg, alkyl ester may be modified.

Preferably, fractional distillation may be carried out by using a charge column type of distillation device. In order to remove low-molecular distillate having a carbon number lower than 20 from the distillate prepared in step b), it is preferable to continuously distillate while changing the requirements such that the concentration of DHA being contained condensates at the top of the column does not exceed lower than 10%, preferably 5%, under the temperature condition between 100 to 200° C. at the bottom of the column, 100 to 180° C. at the top of column, the degree of vacuum between 0.001 to 10 mmHg at the column top, and the reflux ratio of 0.5 to 20, at the distillation column having 5 to 20 steps. At this time, the distillation yield is preferably 50 to 80%.

The charge column is the column being filled with filler therein in order to make the move the material between different phases such as gas and liquid, liquid and liquid, and the like efficient. This is possible by moving the material through, for example, absorption, distillation, adhesion, extraction, and the like efficient by enlarging the contact area between different phases and sufficiently disturbing the flow of each phase.

The present invention preferably uses the filler made of metal, as the filler filling the charge column. The metal filler is used because it is simply made, inexpensive, has low resistance to the gas and large surface area, easy to wet with the solution, light, has sufficient mechanical strength, and excellent heat resistance and corrosion resistance.

By using the charge column type of reduced-pressure fraction device for purifying the omega-3 fatty acid, specific fatty acid components, i.e., eicosapentaenoic acid (EPA), docosahexaenoic acid (DHA) may be continuously produced at the degree of vacuum between 0.001 to 10 mmHg of fatty acid and distillation temperature between 100 to 200° C. Various composition of highly unsaturated fatty acid wherein EPA or DHA purity is 70 to 80% may be produced by changing the distillation requirements according to the carbon number and molecular weight of the objective fatty acids.

The present invention includes the step of purification S500, that is, step e) purifying the concentrated fatty acid from which the saturated fatty acid is removed by means of column chromatography. By step e), the high-purity purification of omega-3 highly unsaturated fatty acid such as eicosapentaenoic acid (EPA), docosahexaenoic acid (DHA), and the like having 90 to 99.7% purity may be completed.

The requirement for high-purity separating DHA is searched by using column chromatography of the reduced-pressure distillation results obtained from step d). A solvent for separating may include acetonitrile and methanol as the main moving phase. The degree of separation is controlled by further adding H₂O such that the solvent is contained in 0.0001˜30 parts by weight, based on 100 parts by weight of the solvent for separating. Thereby, the optimal degree of separation may be set.

A column chromatography may include liquid chromatography (LC), high performance liquid chromatography (HPLC), true moving bed (TMB) or simulated moving bed (SMB) chromatography.

A silicagel or silic acid being coated with AgNO₃ is used as a filler for separating unsaturated fatty acid by using HPLC. Meanwhile, the reverse phase C₁₈ column is economically used, because in the case of separating omega-fatty acid having large molecular weight and lots of double bonds, the separation time is short, and the column is easy to wash with methanol, etc., and may be reused. In addition, it is preferable to use true moving bed chromatography or simulated moving bed chromatography in that it is advantageous for separating the isomer that has similar molecular weight or same molecular weight.

The simulated moving bed column (SMB) is used to effectively separate EPA and DHA, which has similar molecular weight thus has little structural difference, because it characteristically provides high-yield and high-purity separation and the production scale is easy to scale up.

In addition, the simulated moving bed used in the present invention enables the continuous injection of samples and the continuous discharge of products, by connecting multiple chromatography columns to existing liquid chromatography using a single column as a stationary phase, via several types of valves and pumps, so that the separation of a mixture of two or more components, which are difficult to separate or isomers, etc., can be easy, the amount of a solvent used is less than that of existing general chromatography process, and the scale up into commercial scale is easy. Therefore, the simulated moving bed (SMB) in the present invention can prepare omega-3 fatty acid in which EPA coexists with DHA, at the 90% or more, preferably 90 to 99.9% of high purity.

### DESCRIPTION OF DRAWING

FIG. 1 is a flow chart of purification of highly unsaturated fatty acids according to the preferred embodiment of the present invention.

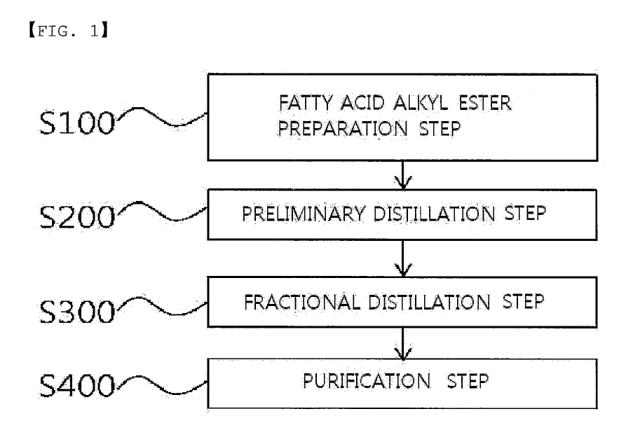

### BEST MODE

Hereinafter, the present invention is further described in detail with reference to the following Examples.

Testing Method
An analysis for the composition and concentration of omega-3 fatty acids used in the present invention was carried out by using HP 6890 series gas chromatography system from the Hewlett-Packard Company, the used column was DB-WAX fused silica capillary column (30 m×0.32 mm×0.25μ). As a detector, FID was used. The temperature of an injector and a detector were 250° C., the temperature of an initial oven was increased from 150° C. to 250° C. (2.5° C./min). As a carrier gas, helium (11 psig) was used.

### Example 1

### a) Preparation of Fatty Acid Alkyl Ester

After the oil or fat, which is tuna oil being subjected to refining, was added to the batch reactor in 1:1 volume ratio with water, Lipase-OF 360,000 (Japan, MEITO SANGYO Company, Triacylglycerol lipase EC 3.1.1.3) extracted from C. rugosa of cylindracea species of Candida genus that is lipase having acyl chain specificity to the triacylglycerol of oil or fat, and Immobilized Lipase (Denmark, Novozyme 435, EC 3.1.1.3) which is lipase acrylic resin extracted from Antarctica species of Candida genus that is lipase having 1,3-positional specificity to triglycerol carbon of oil or fat were respectively added at 38-40° C. in an amount of 3 parts by weight, based on the 100 parts by weight of oil or fat, and hydrolysis was carried out for 24 hrs at an agitating speed of 200 rpm. The spirit ethanol, which was heated to 40° C. 4 hrs later since the initiation of reaction, was added until the molar ratio of oil or fat and ethanol reached 3:1, and the ester exchange reaction was carried out to prepare fatty acid ethyl esters. At this time, the molar ratio of alcohol and oil or fat was kept at 3:1, the reaction time was 48 hrs, and the ester conversion yield was 95% (DHA concentration 25%).

### b) Preliminary Distillation

The fatty acid ethyl ester prepared in the above step a) was continuously distilled by using a centrifugal thin film distillation device or molecular distillation device. At this time, the temperature was 150° C., the degree of vacuum was kept at 0.05 mmHg, and the final recovering yield of the distillate was 55% (DHA concentration 48%).

### c) Reduced-Pressure Fractional Distillation

The distillate prepared in the above step b) was continuously distilled while changing the requirements so that DHA concentration contained in the condensate at the top of the column is 2% or less under the condition of the column bottom temperature is at 180° C., the column top temperature at 150° C., of the degree of vacuum of column top at 5 mmHg, reflux ratio of 5 (reflux ratio=L/D, L=feed amount, D=output amount) at the reduced-pressure distillation column of 15 steps in order to concentrate only EPA or DHA having 20˜22 carbon numbers. At this time, the distillation yield was 73% (DHA concentration 75%).

TABLE 1

| Division | a) | b) | c) |
|---|---|---|---|
| Step | Alcoholysis | Preliminary | Vacuum |
| | Alkyl ester | distillation | fractional |
| | preparation | | distillation |
| Equipment | Supercritical | SPD short- | Reduced- |
| | device | path | pressure |
| | | distillation | distillation |
| | | device | column |
| Max. yield (%) | 95 | 55 | 73 |
| DHA | 25 | 48 | 76 |
| concentration | | | |
| (%) | | | |
| Reaction | 40° C. | 150° C. | 180° C. |
| Temperature | | | |
| Pressure/degree | Normal | 0.005 mmHg | 0.001~1.0 mmHg |
| of vacuum | temperature, | | |
| | Normal | | |
| | pressure | | |
| Cumulated yield | 95% | 52% | 38% |
| (%) | | | |

### d) Purification

The requirement for high-purity separating DHA was searched by using reverse phase column chromatography (Waters, M-501) of the urea-added results obtained from step d). At this time, a solvent for separating includes acetonitrile and methanol as the main mobile phase. The optimal degree of separation may be set by further adding 10 parts by weight of H₂O, based on 100 parts by weight of solvent for separating. The results were represented in the following Table 2.

TABLE 2

| division | d) |
|---|---|
| Analysis condition | Column: μ-bondapak C₁₈ |
| | mobile phase: acetonitrile, |
| | methanol, H₂O |
| | flow rate: 1 ml/min(1000~2000 psi) |
| | Detector: uv 215 nm |
| | Injection volume: 0.5 μl |
| Max. yield (%) | 87 |
| DHA final concentration (%) | 98.7 |

### Example 2

The requirements for scaling up for mass production was searched on the simulated moving bed chromatography (SMB 10 cm×8 columns, Novasep France) based on the results of Example 1. At this time, only methanol was used as a solvent for separating on the main mobile phase. In order to obtain high-purity DHA, the optimized separating requirement was set by varying the aliquot condition. The results were represented in the following Table 3.

TABLE 3
A results analysis table of examination of
SMB aliquot requirements for scale up

| | DHA | Production | Productivity | Concentration |
|---|---|---|---|---|
| Division | purity(%) | yield(%) | (g/day/L) | (%) |
| 1^{st} | 99.49 | 95.83 | 7.4 | 88 |
| 2^{nd} | 99.63 | 89.80 | 6.9 | 81 |
| 3^{rd} | 94.89 | 99.99 | 7.8 | 53 |

In the results of Table 3, DHA purity and production yield shows the inverse proportion relationship. Therefore, if the requirement for separating is set in view of the production yield and concentration, DHA having the desired concentration and yield may be obtained.

The present invention will not be limited by the above-mentioned embodiments and appended drawing. It will be apparent to those skilled in the art that substitution, modifications and variations can be made without departing from the spirit and scope of the invention as defined by the appended claims.

### INDUSTRIAL APPLICABILITY

As set forth above, the high-purity purification method of omega-3 highly unsaturated fatty acid of the present invention has the environmentally friendly effect of minimizing the formation of waste water by adopting the method for preparing fatty acid alkyl ester by alcoholysis by using the lipolysis enzyme, i.e., lipase catalyst and ethanol without using a toxic catalyst or caustic soda, the effect of not generating a cis-trans isomerization reaction in the carbon chain of fatty acid, and the transition reaction of double bonds without using the chemical catalyst and the reaction at high temperature by using an enzyme catalyst, and the effect of forming omega-3 fatty acid keeping the cis structure in the fatty acid.

In addition, the present invention has the effects of high distillation yield in the vacuum fractional distillation process, high productivity, and the removal of pollutant such as heavy metal and PCBs contained in the natural oil or fat, by preliminary concentration process using short-path distillation (SPD) device before the fractional distillation process of fatty acid via the reduced-pressure fractional distillation under the high-vacuum condition of 10⁻³ mmHg or less.

In addition, the present invention has the effect of producing high-purity products by providing the concentration process at 99% or more of DHA concentration via the simulated moving bed chromatography (SMB) purification process using the reduced-pressure fractional distilled DHA concentrate.

In addition, the present invention has the effect of conveniently obtaining high-purity omega-3 highly unsaturated fatty acid having 99% or more purity via a series of continuous processes instead of several steps of separate fractional distillation processes for high-purity concentration.

In addition, the present invention has the effect of obtaining omega-3 highly unsaturated fatty acid to meet the needs according to its use by variously setting the separation condition.

What is claimed is:

1. A high-purity purification method of omega-3 highly unsaturated fatty acid continuously carrying out the following steps, the high-purity purification method comprising:
a) preparing a fatty acid ethyl ester (FAEE) by ethanolysis of a natural oil or fat and ethanol, in the presence of an enzyme catalyst extracted from at least one microorganism selected from the group consisting of Candida genus, Rhizopus genus, Mucor genus, Aspergillus genus and Pseudomonas genus;
b) subjecting the said prepared fatty acid ethyl ester to preliminary distillation using a short-path distillation (SPD) device between 100 and 200° C. and between 0.001 and 10 mmHg;
c) forming a concentrated fatty acid by subjecting the ethyl ester, which has been subjected to the preliminary distillation, to reduced-pressure fractional distillation between 100 and 200° C. and between 0.001 and 10 mmHg; and
d) purifying the concentrated fatty acid by means of simulated moving bed (SMB) column chromatography.

2. The high-purity purification method of omega-3 highly unsaturated fatty acid of claim 1, wherein the enzyme catalyst includes lipase having 1,3-positional specificity to triglycerol carbon of natural oil or fat and lipase having acyl chain specificity to triacylglycerol of natural oil or fat.

3. The high-purity purification method of omega-3 highly unsaturated fatty acid of claim 2, wherein 1,3-positional specificity lipase is at least one lipase selected from the group consisting of Rhizopus javanicus, Rhizopus niveus and Aspergillus niger, and acyl chain specificity lipase is at least one lipase selected from the group consisting of Candida cylindracea, Candida Antarctica, Rhizopus miehei and Rizopus arrhizus.

4. The high-purity purification method of omega-3 highly unsaturated fatty acid of claim 1, wherein the purified omega-3 highly unsaturated fatty acid is EPA (Eicosapentaenoic Acid) or DHA (Docohexaenoic Acid, which has a concentration of 90% or more.
